(19) United States
(12) Patent Application Publication
(10) Pub. No.: US 2021/0093206 A1
(43) Pub. Date: Apr. 1, 2021

(54) APPARATUS FOR DETERMINING BLOOD AND CARDIOVASCULAR CONDITIONS AND METHOD FOR USING THE SAME

(71) Applicant: VITA-COURSE TECHNOLOGIES CO., LTD., Shenzhen (CN)

(72) Inventors: Jiwei ZHAO, Shenzhen (CN); Chuanmin WEI, Shenzhen (CN); Zhiqiang LYU, Shenzhen (CN); Jiantao HAN, Shenzhen (CN); Zhiyong WANG, Shenzhen (CN)

(73) Assignee: VITA-COURSE TECHNOLOGIES CO., LTD., Shenzhen (CN)

(21) Appl. No.: 17/120,091

(22) Filed: Dec. 11, 2020

Related U.S. Application Data
(63) Continuation of application No. PCT/CN2018/090748, filed on Jun. 12, 2018.

(51) Int. Cl. A61B 5/0205; A61B 5/28; A61B 5/00; A61B 5/339; A61B 5/02; A61B 5/1455; A61B 5/145

(52) U.S. Cl. CPC A61B 5/02055; A61B 5/28; A61B 5/6826; A61B 5/339; A61B 5/02108; A61B 5/14551; A61B 5/14532; A61B 5/02007; A61B 5/0006; A61B 5/02035

(57) ABSTRACT

A blood and cardiovascular condition determination apparatus is provided. The blood and cardiovascular condition determination apparatus may include a first pulse wave signal sensor, a second pulse wave signal and a processing unit. The first pulse wave signal sensor may be configured to generate a first pulse wave signal associated with a first section of a living body. The second pulse wave signal sensor may be configured to generate a second pulse wave signal associated with a second section of the living body. The processing unit may determine at least one blood and cardiovascular condition based on the first pulse wave signal and the second pulse wave signal. The at least one blood and cardiovascular condition may include at least one of a blood pressure, a blood sugar level, a blood oxygen level, a blood vessel aging level, or a blood viscosity.

### CROSS-REFERENCE TO RELATED APPLICATION

This application is a continuation of International Application No. PCT/CN2018/090748 filed on Jun. 12, 2018, the entire contents of which are hereby incorporated by reference.

### TECHNICAL FIELD

The present disclosure generally relates to the determination of blood and cardiovascular conditions, and in particular, to systems, methods and apparatuses for determining blood and cardiovascular conditions based on pulse wave signals.

### BACKGROUND

Heart disease is the leading cause of death in the United States, accounting for six hundred thousand deaths per year. Heart disease also costs the United States about $200 billion each year, including the cost of health care services, medications, and lost productivity. High blood pressure (or hypertension) and high blood sugar (or hyperglycemia) are the most two common risk factors for heart disease and nothing would save more lives than getting blood pressure and blood sugar under control.

Beside the blood pressure and the blood sugar, other blood and cardiovascular conditions, such as blood oxygen level, blood vessel aging level, or blood viscosity are also related to heart disease. However, when the values related to those blood and cardiovascular conditions fluctuate that may cause life threatening danger, the symptoms is usually hard to be detected immediately. Therefore, the key to prevent sudden heart disease-related deaths may be by monitoring of these blood and cardiovascular conditions. It is desirable to provide methods and systems for efficiently determining the blood and cardiovascular conditions.

### SUMMARY

In an aspect of the present disclosure, a blood and cardiovascular condition determination apparatus is provided. The blood and cardiovascular condition determination apparatus may include a first pulse wave signal sensor, a second pulse wave signal and a processing unit. The first pulse wave signal sensor may be configured to generate a first pulse wave signal. The first pulse wave signal sensor may include a first light emitter configured to emit a first light signal to a first section of a living body, wherein the first light signal is reflected by the first section. The first pulse wave signal sensor may further include a first light receiver configured to receive the reflected first light signal and generate the first pulse wave signal based on the reflected first light signal. The second pulse wave signal sensor may be configured to generate a second pulse wave signal. The second pulse wave signal sensor may include a second light emitter configured to emit a second light signal to a second section of the living body, wherein the second light signal is reflected by the second section. The second pulse wave signal sensor may further include a second light receiver configured to receive the reflected second light signal and generate the second pulse wave signal based on the reflected second light signal. The processing unit may be configured to determine at least one blood and cardiovascular condition of the living body based on the first pulse wave signal and the second pulse wave signal.

In some embodiments, the at least one blood and cardiovascular condition may include at least one of a blood pressure, a blood sugar level, a blood oxygen level, a blood vessel aging level, or a blood viscosity.

In some embodiments, the first light signal may be configured to determine a first blood and cardiovascular condition of the living body, and the second light signal may be configured to determine a second blood and cardiovascular condition of the living body.

In some embodiments, the first light signal may be configured to have higher intensity than the second light signal. The processing unit may be further configured to determine at least one first segment in the first pulse wave that is saturated and at least one corresponding second segment in the second pulse wave, wherein the at least one corresponding second segment is at a same time period as the at least one first segment, respectively. The processing unit may be further configured to replace the at least one first segment in the first pulse wave by the at least one corresponding second segment to generate a combined pulse wave signal. The processing unit may be further configured to determine the at least one blood and cardiovascular condition of the living body based on the combined pulse wave signal.

In some embodiments, the processing unit may be further directed to determine an average pulse wave signal based on the first pulse wave signal and the second pulse wave signal, and at least one blood and cardiovascular condition of the living body based on the average pulse wave signal.

In some embodiments, the processing unit may be directed to determine a first signal-to-noise ratio (SNR) of the first pulse wave signal and a second SNR of the second pulse wave signal. The processing unit may be further directed to determine a first weight of the first pulse wave signal and a second weight of the second pulse wave signal based on the first SNR and the second SNR. The processing unit may be further configured to determine the average pulse wave signal based on the first pulse wave signal, the second pulse wave signal, the first weight, and the second weight.

In some embodiments, the first light signal may include at least two different wavelengths.

In some embodiments, the blood and cardiovascular condition determination apparatus may further include a temperature sensor configured to obtain a temperature of the first section when the first light signal is received. The processing unit may be further configured to update the first pulse wave signal based on the temperature of the first tissue section to generate an updated first pulse wave signal and determine the at least one blood and cardiovascular condition of the living body based on the updated first pulse wave signal and the second pulse wave signal.

In some embodiments, the blood and cardiovascular condition determination apparatus may further include a temperature controller configured to maintain the temperature of the first section of the living body.

In some embodiments, the blood and cardiovascular condition determination apparatus may further include a motion sensor configured to obtain a motion of the first section when the first light signal is received. The processing unit may be further configured to update the first pulse wave signal based on the motion of the first section to generate an updated first pulse wave signal. The processing unit may be further configured to determine at least one blood and cardiovascular condition of the living body based on the updated first pulse wave signal and the second pulse wave signal.

In some embodiments, the blood and cardiovascular condition determination apparatus may further include an ECG electrode configured to acquire a biopotential signal of the living body. The processing unit may be further configured to determine the at least one blood and cardiovascular condition of the living body based on the first pulse wave signal, the second pulse wave signal, and the biopotential signal.

In some embodiments, at least one of the first pulse wave signal sensor or the second pulse wave signal sensor may be implemented on a wearable device attached to a finger of a human body.

In some embodiments, the blood and cardiovascular condition determination apparatus may further include a screen configured to display the at least one blood and cardiovascular condition.

In some embodiments, the blood and cardiovascular condition determination apparatus may further include a transceiver configured to transmit the at least one blood and cardiovascular condition to an electronic device.

In another aspect of the present disclosure, a blood and cardiovascular condition determination method is provided. The blood and cardiovascular condition determination method may be implemented on a computing device having at least one processor, at least one computer-readable storage medium, and a communication platform connected to a network. The blood and cardiovascular condition determination method may include generating, by a first pulse wave signal sensor, a first pulse wave signal. The generation of the first pulse wave signal may include emitting, by a first light emitter, a first light signal to a first section of a living body, wherein the first light signal is reflected by a first section, and receiving, by a first light receiver, the reflected first light signal and generating the first pulse wave signal based on the reflected first light signal. The blood and cardiovascular condition determination method may further include generating, by a second pulse wave signal sensor, a second pulse wave signal. The generation of the second pulse wave signal may include emitting, by a second light emitter, a second light signal to a second section of a living body, wherein the second light signal is reflected by a second section, and receiving, by a second light receiver, the reflected second light signal and generating the second pulse wave signal based on the reflected second light signal. The blood and cardiovascular condition determination method may further include determining, by a processing unit, at least one blood and cardiovascular condition of the living body based on the first pulse wave signal and the second pulse wave signal.

In yet a further aspect of the present disclosure, a non-transitory computer-readable storage medium storing instructions is provided. When executed by at least one processor of a system, the instructions may cause the system to perform a blood and cardiovascular condition determination method. The blood and cardiovascular condition determination method may include generating, by a first pulse wave signal sensor, a first pulse wave signal. The generation of the first pulse wave signal may include emitting, by a first light emitter, a first light signal to a first section of a living body, wherein the first light signal is reflected by a first section, and receiving, by a first light receiver, the reflected first light signal and generating the first pulse wave signal based on the reflected first light signal. The blood and cardiovascular condition determination method may further include generating, by a second pulse wave signal sensor, a second pulse wave signal. The generation of the second pulse wave signal may include emitting, by a second light emitter, a second light signal to a second section of a living body, wherein the second light signal is reflected by a second section, and receiving, by a second light receiver, the reflected second light signal and generating the second pulse wave signal based on the reflected second light signal. The blood and cardiovascular condition determination method may further include determining, by a processing unit, at least one blood and cardiovascular condition of the living body based on the first pulse wave signal and the second pulse wave signal.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary blood and cardiovascular condition determination system according to some embodiments of the present disclosure;

FIG. 2 is a schematic diagram illustrating exemplary hardware and/or software components of a computing device according to some embodiments of the present disclosure;

FIG. 3 is a schematic diagram illustrating exemplary hardware and/or software components of a mobile device on which a terminal may be implemented according to some embodiments of the present disclosure;

FIG. 4 is a schematic diagram illustrating an exemplary blood and cardiovascular condition determination apparatus according to some embodiments of the present disclosure;

FIG. 5 is a block diagram illustrating an exemplary data processing device according to some embodiments of the present disclosure;

FIG. 6 is a flowchart illustrating an exemplary process for determining blood and cardiovascular condition according to some embodiments of the present disclosure;

FIG. 7 is a schematic diagram illustrating an exemplary process for generating a combined pulse wave signal according to some embodiments of the present disclosure;

FIG. 8 is a flowchart illustrating an exemplary process for updating a first pulse wave signal according to some embodiments of the present disclosure;

FIG. 9 is a schematic diagram illustrating an exemplary method for generating a combined pulse wave signal according to some embodiments of the present disclosure;

FIG. 10A is a schematic diagram illustrating a perspective view of an exemplary blood and cardiovascular condition determination apparatus according to some embodiments of the present disclosure;

FIG. 10B is a diagram illustrating a bottom view of an exemplary blood and cardiovascular condition determination apparatus according to some embodiments of the present disclosure;

FIG. 11A is a schematic diagram illustrating lateral view of an exemplary blood and cardiovascular condition determination apparatus according to some embodiments of the present disclosure;

FIG. 11B is a diagram illustrating a side view of an exemplary blood and cardiovascular condition determination apparatus according to some embodiments of the present disclosure; and.

FIG. 12 is a diagram illustrating wearing view of an exemplary blood and cardiovascular condition determination apparatus according to some embodiments of the present disclosure.

### DETAILED DESCRIPTION

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown but is to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or other storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in a firmware, such as an erasable programmable read-only memory (EPROM). It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The flowcharts used in the present disclosure illustrate operations that systems implement according to some embodiments in the present disclosure. It is to be expressly understood, the operations of the flowchart may be implemented not in order. Conversely, the operations may be implemented in inverted order, or simultaneously. Moreover, one or more other operations may be added to the flowcharts. One or more operations may be removed from the flowcharts.

Moreover, while the systems, methods, and/or apparatuses disclosed in the present disclosure are described primarily regarding the determination of blood and cardiovascular conditions, it should also be understood that this is only one exemplary embodiment. The systems, methods, and/or apparatuses of the present disclosure may be applied to the determination of any other kind of heath conditions (e.g. body temperature, body fat percentage, hepatobiliary function, thyroid function, bone density).

The term “living body” in the present disclosure may refer to an individual that may be detected in the blood and cardiovascular condition determination system. For example, the living body may be a human, an animal, a section of the human (e.g., finger, wrist, arm, head, heart,), or the like, or a combination thereof.

An aspect of the present disclosure relates to systems, methods, and apparatuses for blood and cardiovascular condition determination. The apparatus may include a first pulse wave signal sensor, a second pulse wave signal sensor and a processor (or microcontroller unit, MCU). The first pulse wave signal sensor and the second pulse wave signal sensor may each include a light emitter and a light receiver. The light emitter may emit a light to a section of a living body and the light may be reflected by the section of the living body. The reflected light may be obtained by the light receiver to generate a pulse wave signal. For example, the first pulse wave signal sensor may generate a first pulse wave signal of a first section of the living body and the second pulse wave signal sensor may generate a second pulse wave signal of a second section of the living body. The processor may determine at least one blood and cardiovascular condition of the living body based on the first pulse wave signal and the second pulse wave signal. For example, the first pulse wave signal and the second pulse wave signal may both be related to blood pressure and the processor may determine the blood pressure of the living body by taking an average of the first pulse wave signal and the second pulse wave signal. As used herein, “be related to” blood pressure means the light emitted by the light emitter is at certain penetration characteristics (e.g., intensity, wavelength, diffusion angle) such that the blood pressure can be obtained according to the first pulse wave signal. Different blood and cardiovascular conditions may require different penetration characteristics. As another example, the first pulse wave signal may be related to blood pressure, and the second pulse wave signal may be related to blood oxygen level. The processor may generate the blood pressure and the blood oxygen level of the living body based on the first pulse wave signal and the second pulse wave signal. In some embodiments, one of the first pulse wave signal sensor and the second pulse wave signal sensor may be a main sensor while the other may be a supplementary sensor. The main sensor generally has higher SNR but is easy to be saturated (e.g., reaches a maximum value due to design limitations of the sensor). When the main sensor is not saturated, the pulse wave signal generated by the main sensor may be used to determine the at least one blood and cardiovascular condition. When the main sensor is saturated, the pulse wave signals generated by the two sensors may be combined to generate a combined pulse wave signal. The at least one blood and cardiovascular condition may be determined by the combined pulse wave signal.

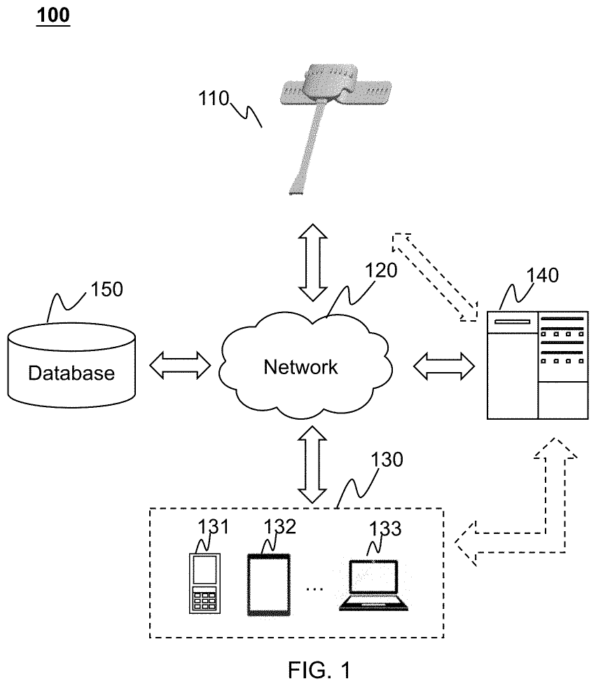

FIG. 1 is a schematic diagram illustrating an exemplary blood and cardiovascular condition determination system according to some embodiments of the present disclosure. As shown in FIG. 1, the blood and cardiovascular condition determination system 100 may include a blood and cardiovascular condition determination apparatus 110, a network 120, a terminal 130, a server 140, and a database 150.

In some embodiments, the blood and cardiovascular condition determination apparatus 110 may be sphygmomanometer, glucose meter, a pulse oximetry, or the like, or any combination thereof. In some embodiments, the blood and cardiovascular condition determination apparatus 110 may be any other kind of heath condition determination apparatus (e.g. a temperature sensor, a body fat percentage detection apparatus, a hepatobiliary scan). The blood and cardiovascular condition determination apparatus 110 may include a plurality of sensors configured to obtain various kinds of information of the living body. The plurality of sensors may include but not limited to a pulse wave signal sensor, a temperature sensor, a motion sensor, or the like, or any combination thereof. The blood and cardiovascular condition determination apparatus 110 may also include a temperature controller (e.g., a metal electrode), and/or an electrocardiogram electrode. The plurality of sensors, the temperature controller and/or the electrocardiogram (ECG) electrode may be configured on a side of the blood and cardiovascular condition determination apparatus 110, via which, the blood and cardiovascular condition determination apparatus 110 is further attached to a part of a living body. In some embodiments, the blood and cardiovascular condition determination apparatus 110 is wrapped around and tightened against a section of a living body where an artery can be sensed.

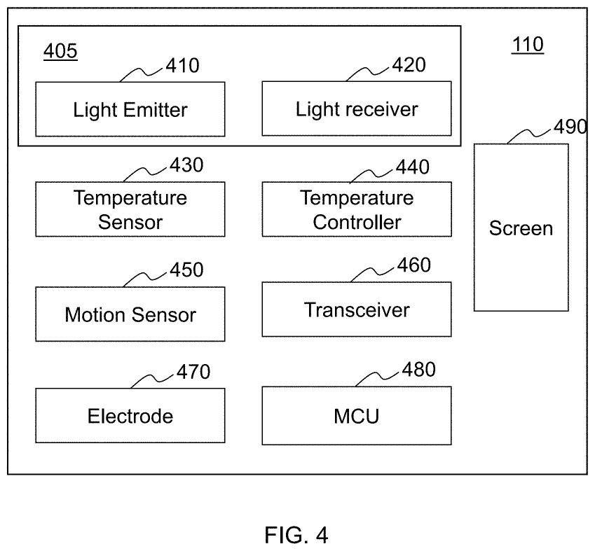

For example, the blood and cardiovascular condition determination apparatus 110 may include a strap, and the sensors, the temperature controller and/or the ECG electrode may be configured on the inner side of the strap. The sensors, the temperature controller and/or the ECG electrode may be manufactured as part of the blood and cardiovascular condition determination apparatus 110. Alternatively, the sensors, the temperature controller and/or the ECG electrode may be detachable from the blood and cardiovascular condition determination apparatus 110. The strap may be wrapped around a finger (as a fingerstall), a wrist, a forearm, an upper arm, a torso, an upper leg, a lower leg, a toe, or an ankle of the living body. Merely by way of example, the blood and cardiovascular condition determination apparatus 110 may include two (or even more) similar or identical straps (carrying the sensors) being wrapped around two sections of the living body (e.g., two fingers, a finger and a toe). In some embodiments, the blood and cardiovascular condition determination apparatus 110 may include a processor (or MCU) configured to process the signals received from the sensors and the ECG electrode to generate the at least one blood and cardiovascular condition of the living body. Detailed descriptions of the exemplary blood and cardiovascular condition determination apparatus may be found elsewhere in the present disclosure (e.g., FIGS. 4, 10A, 10B, 11A, 11B, 12 and the descriptions thereof).

The network 120 may facilitate exchange of information and/or data for the blood and cardiovascular condition determination system 100. In some embodiments, one or more components of the blood and cardiovascular condition determination system 100 (e.g., the blood and cardiovascular condition determination apparatus 110, the terminal 130, the server 140, the database 150, etc.) may communicate information and/or data with one or more other components of the blood and cardiovascular condition determination system 100 via the network 120. For example, the server 140 may obtain pulse wave signals, temperature signals, motion signals and/or ECG signals from the corresponding component of the blood and cardiovascular condition determination apparatus 110 via the network 120. As another example, the server 140 may obtain user instructions from the terminal 130 via the network 120.

The network 120 may be and/or include a public network (e.g., the Internet), a private network (e.g., a local area network (LAN), a wide area network (WAN)), etc.), a wired network (e.g., an Ethernet network), a wireless network (e.g., an 802.11 network, a Wi-Fi network, etc.), a cellular network (e.g., a Long Term Evolution (LTE) network), a frame relay network, a virtual private network (“VPN”), a satellite network, a telephone network, routers, hubs, switches, server computers, and/or any combination thereof. Merely by way of example, the network 120 may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network 120 may include one or more network access points. For example, the network 120 may include wired and/or wireless network access points such as base stations and/or internet exchange points through which one or more components of the blood and cardiovascular condition determination system 100 may be connected to the network 120 to exchange data and/or information.

The terminal(s) 130 may communicate with the blood and cardiovascular condition determination apparatus 110, and/or the server 140. For example, a user may set a parameter (e.g., a type of blood and cardiovascular condition to be monitored, an age, a height, a weight of the living body) via an input device (e.g., a keyboard, a touch screen) of the terminal 130. The terminal 130 may transmit the parameter to the blood and cardiovascular condition determination apparatus 110 via the network 120. As another example, the terminal 130 may obtain a determination result of the blood and cardiovascular conditions of a living body from the server 140 or the blood and cardiovascular condition determination apparatus 110. The determination result may be displayed on the graphic user interface (GUI) of the terminal 130. The terminal 130 may include a mobile device 131, a tablet computer 132, a laptop computer 133, or the like, or any combination thereof. In some embodiments, the mobile device 131 may include a smart home device, a wearable device, a mobile device, a virtual reality device, an augmented reality device, or the like, or any combination thereof. In some embodiments, the smart home device may include a smart lighting device, a control device of an intelligent electrical apparatus, a smart monitoring device, a smart television, a smart video camera, an interphone, or the like, or any combination thereof. In some embodiments, the wearable device may include a bracelet, a footgear, eyeglasses, a helmet, a watch, clothing, a backpack, a smart accessory, or the like, or any combination thereof. In some embodiments, the mobile device may include a mobile phone, a personal digital assistance (PDA), a gaming device, a navigation device, a point of sale (POS) device, a laptop, a tablet computer, a desktop, or the like, or any combination thereof. In some embodiments, the virtual reality device and/or the augmented reality device may include a virtual reality helmet, virtual reality glasses, a virtual reality patch, an augmented reality helmet, augmented reality glasses, an augmented reality patch, or the like, or any combination thereof. For example, the virtual reality device and/or the augmented reality device may include a Google Glass™, an Oculus Rift™, a Hololens™, a Gear VR™, etc. In some embodiments, the terminal(s) 130 may be part of the server 140.

The server 140 may process data and/or information obtained from the blood and cardiovascular condition determination apparatus 110, the terminal 130, and/or the database 150. For example, the server 140 may process signals obtained from the blood and cardiovascular condition determination apparatus 110 and determine at least one blood and cardiovascular condition of the living body (e.g., a blood pressure). In some embodiments, the server 140 may be a computer, a user console, a single server or a server group, etc. The server group may be centralized or distributed. In some embodiments, the server 140 may be local or remote. For example, the server 140 may access information and/or data stored in the blood and cardiovascular condition determination apparatus 110, the terminal 130, and/or the database 150 via the network 120. As another example, the server 140 may be directly connected to the blood and cardiovascular condition determination apparatus 110, the terminal 130 and/or the database 150 to access stored information and/or data. In some embodiments, the server 140 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof. In some embodiments, some or all of the tasks of the server 140 may be allocated to the processor in the blood and cardiovascular condition determination apparatus 110. For example, the server 140 may be omitted and the pulse wave signal, the temperature signal, and/or the motion signal may be directly processed by the MCU in the blood and cardiovascular condition determination apparatus 110 to generate the at least one blood and cardiovascular condition of the living body. As another example, the processor in the blood and cardiovascular condition determination apparatus 110 may process the pulse wave signal while the server 140 may update the pulse wave signal based on the temperature and/or motion information and determine the at least one blood and cardiovascular condition of the living body based on the updated pulse wave signal.

The database 150 may store data, instructions, and/or any other information. In some embodiments, the database 150 may store data obtained from the blood and cardiovascular condition determination apparatus 110, the terminal 130 and/or the server 140. In some embodiments, the database 150 may store data and/or instructions that the server 140 may execute or use to perform exemplary methods described in the present disclosure. In some embodiments, the database 150 may include a mass storage, a removable storage, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memory may include a random access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (EPROM), an electrically erasable programmable ROM (EEPROM), a compact disk ROM (CD-ROM), and a digital versatile disk ROM, etc. In some embodiments, the database 150 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the database 150 may be connected to the network 120 to communicate with one or more other components in the blood and cardiovascular condition determination system 100 (e.g., the blood and cardiovascular condition determination apparatus 110, the server 140, the terminal 130, etc.). One or more components in the blood and cardiovascular condition determination system 100 may access the data or instructions stored in the database 150 via the network 120. In some embodiments, the database 150 may be directly connected to or communicate with one or more other components in the blood and cardiovascular condition determination system 100 (e.g., the blood and cardiovascular condition determination apparatus 110, the server 140, the terminal 130, etc.). In some embodiments, the database 150 may be part of the blood and cardiovascular condition determination apparatus 110, or the server 140.

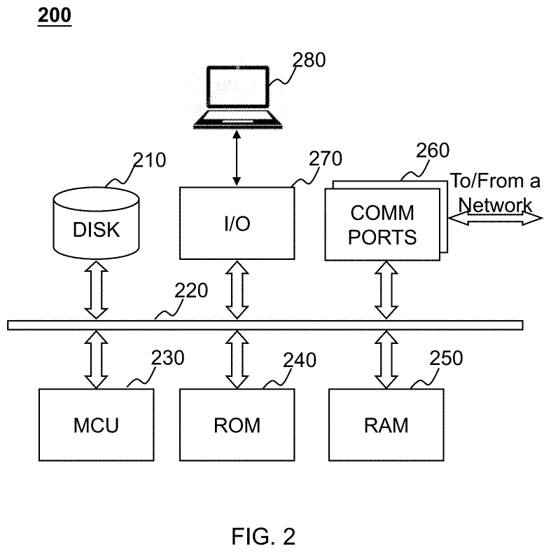

FIG. 2 is a schematic diagram illustrating exemplary hardware and software components of a computing device 200 on which server 140 or a portion thereof may be implemented according to some embodiments of the present disclosure. For example, the server 140 may be implemented on the computing device 200 and configured to perform functions of the server 140 described in this disclosure.

The computing device 200 may be a general-purpose computer or a special purpose computer, both may be used to implement a blood and cardiovascular condition determination system 100 for the present disclosure. The computing device 200 may be used to implement any component for signal processing as described herein. For example, the server 140 may be implemented on the computing device 200, via its hardware, software program, firmware, or any combination thereof. Although only one such computer is shown, for convenience, the computer functions relating to the data processing as described herein may be implemented in a distributed fashion on a number of similar platforms, to distribute the processing load.

The computing device 200, for example, may include communication (COMM) ports 260 connected to and from a network to facilitate data communications. The computing device 200 may also include a processor 230 (e.g., a microcontroller unit (MCU)), in the form of one or more processors, for executing program instructions. The exemplary computer platform may include an internal communication bus 220, program storage and data storage of different forms, for example, a disk 210, and a read only memory (ROM) 240, or a random-access memory (RAM) 250, for various data files to be processed and/or transmitted by the computer. The exemplary computer platform may also include program instructions stored in the ROM 240, RAM 250, and/or another type of non-transitory storage medium to be executed by the processor 230. The methods and/or processes of the present disclosure may be implemented as the program instructions. The computing device 200 also includes an I/O component 270, supporting input/output between the computer and other components therein such as user interface elements 280. The computing device 200 may also receive programming and data via network communications.

Merely for illustration, only one processor is illustrated in the computing device 200. However, it should be noted that the computing device 200 in the present disclosure may also include multiple processors, thus operations and/or method steps that are performed by one processor as described in the present disclosure may also be jointly or separately performed by the multiple processors. For example, if in the present disclosure the processor of the computing device 200 executes both operation A and operation B, it should be understood that operation A and operation B may also be performed by two different processors jointly or separately in the computing device 200 (e.g., the first processor executes operation A and the second processor executes operation B, or the first and second processors jointly execute operations A and B).

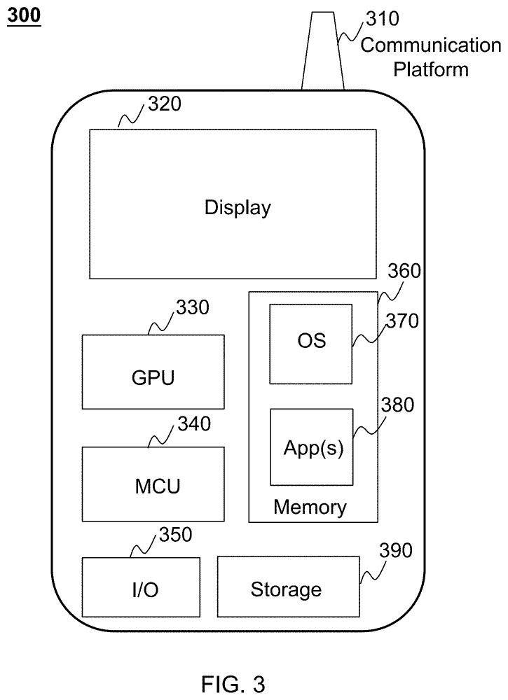

FIG. 3 is a schematic diagram illustrating exemplary hardware and/or software components of an exemplary mobile device 300 on which a terminal 130 may be implemented according to some embodiments of the present disclosure. As illustrated in FIG. 3, the mobile device 300 may include a communication platform 310, a display 320, a graphic processing unit (GPU) 330, a microcontroller unit (MCU) 340, an I/O 350, a memory 360, an operation system (OS) 370, applications 380, and storage 390. In some embodiments, any other suitable component, including but not limited to a system bus or a controller (not shown), may also be included in the mobile device 300. In some embodiments, a mobile operating system 370 (e.g., iOS™, Android™, Windows Phone™, etc.) and one or more applications 380 may be loaded into the memory 360 from the storage 390 in order to be executed by the MCU 340. The applications 380 may include a browser or any other suitable mobile apps for receiving and rendering information relating to signal processing or other information from the server 140. The application 380 may be a blood and cardiovascular condition determination application working cooperatively with the blood and cardiovascular condition determination apparatus 110 and the server 140. The data related to the blood and cardiovascular condition determination application, i.e., the application 380, may be stored in the storage 390 of the mobile device 300 and synchronized to the blood and cardiovascular condition determination apparatus 110 and the server 140 via the network 120 (as illustrated in FIG. 1). For example, a user may select a blood and cardiovascular condition (e.g., blood pressure, blood sugar) to be monitored via the application 380 installed on the mobile device 300. The selection may be simultaneously transmitted to the blood and cardiovascular condition determination apparatus 110 and the server 140 via the network 120. The blood and cardiovascular condition determination apparatus 110 may obtain signals related to the blood and cardiovascular condition and the server 140 or the processor in the blood and cardiovascular condition determination apparatus 110 may determine the blood and cardiovascular condition of the living body based on the signals. The blood and cardiovascular condition may be displayed to the user in the application 380 on the display 320 via a GUI of the mobile device 300. User interactions with the information stream may be achieved via the I/O 350 and provided to the server 140 and/or other components of the blood and cardiovascular condition determination system 100 via the network 120.

FIG. 4 is a schematic diagram illustrating an exemplary blood and cardiovascular condition determination apparatus according to some embodiments of the present disclosure. As shown in FIG. 4, the blood and cardiovascular condition determination apparatus 110 may include a pulse wave signal sensor 405, a temperature sensor 430, a temperature controller 440, a motion sensor 450, a transceiver 460, an electrode 470, a MCU 480 and a screen 490.

The pulse wave signal sensor 405 may include a light emitter 410 and a light receiver 420. The light emitter 410 may be configured to emit a light signal to a section of a living body. The light signal may penetrate through or be reflected by the section of the living body. The light signal emitted by the light emitter 410 may have different penetration characteristics. The penetration characteristics may include a wavelength, a diffusion angle, penetration depth, or the like, or any combination thereof. For example, the light emitter 410 may emit the light signal with an infrared wavelength (e.g., 700 nm-1 mm) at a small diffusion angle. Different blood and cardiovascular conditions may have different requirements on the penetration characteristics. In some embodiments, the light emitter 410 may be a laser diode (LD) or a light emitting diode (LED).

The light receiver 420 may be configured to receive the reflected or penetrated light signal and generate a pulse wave signal. More particularly, the light receiver 420 may perform a photoelectric conversion of the signal that converts an intensity of the reflected light signal to a voltage or current of the pulse wave signal. In some embodiments, the blood and cardiovascular condition determination apparatus 110 may include two or more pulse wave sensors (two or more pairs of light emitters 410 and light receivers 420).

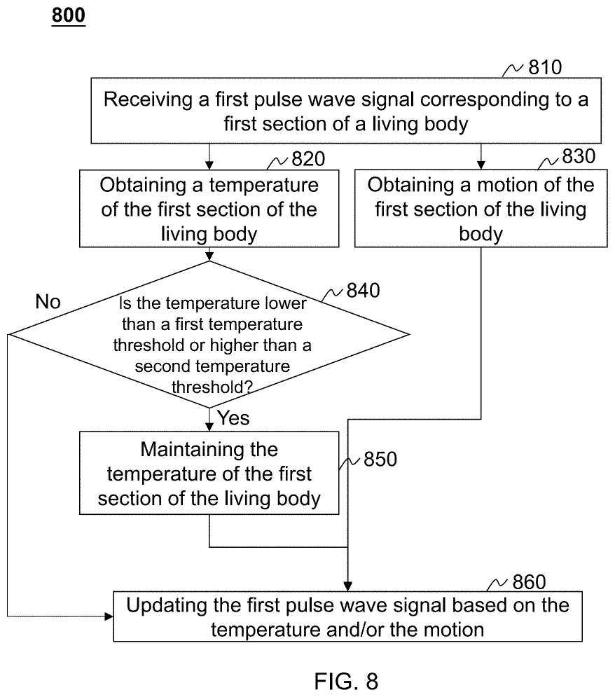

The temperature sensor 430 may be configured to measure a temperature of the living body. The blood and cardiovascular condition is normally determined by assuming the temperature of the living body is at 36.5° C. However, the real temperature measured from the section of the living body may be slightly different from the body temperature. For example, the temperature measured from the forehead is slightly lower than the temperature measured from the armpit. As another example, the real temperature of the section of the living body may also depend on the room temperature. The blood and cardiovascular condition may be updated (or referred to as “corrected” or “compensated”) based on the real temperature of the section of the living body and a relationship between temperature and blood and cardiovascular condition or a temperature difference between the 36.5° C. and the real temperature of the section of the living body and the blood and cardiovascular condition. In some embodiments, the pulse wave signal is updated or corrected based on the real temperature and a relationship between temperature and pulse wave signal. The updated or corrected pulse wave signal may be used to determine the blood and cardiovascular condition. Detailed descriptions of the corrections of the pulse wave signal may be found elsewhere in this disclosure (e.g., FIG. 8 and the descriptions thereof).

The temperature controller 440 may be configured to maintain the temperature of the section of the living body. In some embodiments, the temperature controller 440 may maintain the temperature of the section at a constant value or within a predetermined range during the whole detection process. In some embodiments, upon detecting the temperature of the section is lower than a first temperature threshold (e.g., 20° C.) or greater than a second temperature threshold (e.g., 45° C.), the temperature controller 440 may be activated in order to maintain the temperature of the section. In some embodiments, the temperature controller 440 may be a thermostat (e.g., a bimetal thermostat), a metal electrode, or the like, or any combination thereof.

The motion sensor 450 may be an accelerometer, gyroscope, gradiometer, or any other motion sensing device. In some embodiments, the motion sensor 450 may be configured to detect a motion of the section of the living body. The motion of the section may include but not limited to moving from side to side, tilting upward and downward, tremoring, etc. and may be depicted by various parameters including a distance, a speed, a direction, an intensity, and/or a moving trace. The motion sensor 450 may detect the motion of the section in one direction or in multiple directions. A three-axis accelerometer, for instance, may output accelerations of the section of the living body in x, y, and z directions.

In some embodiments, the motion sensor 450 may be configured to obtain the motion of the section of the living body when a light signal is emitted by the light emitter 410 or received by the light receiver 420. The blood and cardiovascular condition may be updated or corrected based on the motion of the section of the living body and a relationship between motion and blood and cardiovascular condition. In some embodiments, the pulse wave signal may be updated or corrected based on the motion of the section and a relationship between motion and pulse wave signal. The updated or corrected pulse wave signal may be used to determine the blood and cardiovascular condition. Detailed descriptions of the corrections of the pulse wave signal may be found elsewhere in this disclosure (e.g., FIG. 8 and the descriptions thereof).

The transceiver 460 may be configured to transmit information to or receive information from the terminal 130 and/or the server 140. The information may be a pulse wave signal, a temperature signal, a motion signal, an ECG signal, a determined blood and cardiovascular condition, a healthy index, or the like, or any combination thereof. In some embodiments, the transceiver 460 may be a Fiber Optical Transceiver (FOT).

The electrode 470 may be configured to capture ECG signal of the section of the living body. The ECG signal may be evaluated solely or in combination with the pulse wave signal to determine at least one blood and cardiovascular condition or heart condition of the living body.

The microcontroller unit (MCU or processor) 480 may process signals received by the light receiver 420, temperature sensor 430, the motion sensor 450, and/or the electrode 470 and determine at least one blood and cardiovascular condition of the living body based on the signals.

In some embodiments, some or all of the functions of the MCU 480 may be implemented on the server 140, and thus, the function of the MCU 480 may be simplified or the MCU 480 may be eliminated. The signals received by the light receiver 420, temperature sensor 430, the motion sensor 450, and/or the electrode 470 may be sent to the server 140 to be further processed. As another example, the MCU 480 may process the pulse wave signal while the server 140 may update the pulse wave signal based on the temperature and/or motion signal.

The screen 490 may display information. The information displayed by the screen may be received from the light receiver 420, the temperature sensor 430, the motion sensor 450, the electrode, and/or the MCU 480. For example, the screen 490 may display a pulse wave signal of the living body as detected in real-time. As another example, the screen 490 may display an animation of the motion of the living body. The information displayed may be in a form of a text, a sound, an image, a video, or the like, or any combination thereof. In some embodiments, the screen 490 may display one or more blood and cardiovascular conditions of the living body. For example, the screen 490 may display that the diastolic pressure of the living body is 70 mmHg and the systolic pressure of the living body is 120 mmHg. As another example, the screen 490 may display that the blood sugar level of the living body is 5 mmol/L and the blood oxygen level of the living body is 95%. As another example, the screen 490 may display the blood pressure and the blood sugar level in combination with the animated motion of the living body. It should be noted that some of the functions of the screen 490 may be realized by the display 320 of the mobile device 300 (or terminal 130). For example, if the screen 490 is omitted in the blood and cardiovascular condition determination apparatus, the information may be displayed on the display 320 of the mobile device 300 (or terminal 130).

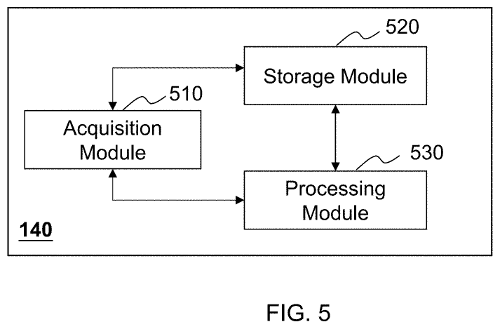

FIG. 5 is a block diagram illustrating an exemplary server 140 according to some embodiments of the present disclosure. As shown in FIG. 5, the server 140 may include an acquisition module 510, a storage module 520, and a processing module 530. At least a portion of the server 140 may be implemented on the computing device 200 as illustrated in FIG. 2, or the mobile device 300 as illustrated in FIG. 3.

The acquisition module 510 may acquire data. The data may be acquired from one or more components of the blood and cardiovascular condition determination system 100, such as the blood and cardiovascular condition determination apparatus 110. In some embodiments, the data may be acquired from an external data source via the network 120. The data acquired may include information regarding a living body or a section of the living body (e.g., the finger, the wrist, the arm, the heart). For example, the data acquired may be a pulse wave signal corresponding to the blood pressure of the living body. In some embodiments, the acquisition module 510 may include a wireless transceiver to receive the information via the network 120.

The storage module 520 may store data. The data stored may be a numerical value, a signal, an image, information of a living body, an instruction, an algorithm, or the like, or a combination thereof. The data stored may be acquired by the acquisition module 510, imported via the terminal 130, generated in the processing module 530, or pre-stored in the storage module 520 during system initialization or before an operation of data processing. The storage module 520 may include a system storage device (e.g., a disk) that is provided integrally (i.e. substantially non-removable), or a storage device that is removable connectable to the system via, for example, a port (e.g., a UBS port, a firewire port, etc.), a drive (a disk drive, etc.), etc. The storage module 520 may include, for example, a hard disk, a floppy disk, selectron storage, random access memory (RAM), dynamic random access memory (DRAM), static random access memory (SRAM), bubble memory, thin film memory, magnetic plated wire memory, phase change memory, flash memory, a cloud disk, or the like, or a combination thereof. The storage module 520 may be connected to or communicate with the acquisition module 510 and/or the processing module 530. In some embodiments, the storage module 520 may be operationally connected with one or more virtual storage resources (e.g., cloud storage, a virtual private network, other virtual storage resources, etc.) via the network 120.

The processing module 530 may process data and determine at least one blood and cardiovascular condition of the living body. The data may be acquired from the acquisition module 510, the storage module 520, etc. In some embodiments, the data processed may be acquired from an external data source via the network 120. For example, the processing module 530 may obtain a pulse wave signal, a temperature signal, and a motion signal from the acquisition module 510. The processing module 530 may update the pulse wave signal based on the temperature signal and/or the motion signal to generate an updated pulse wave signal. The processing module 530 may determine the at least one blood and cardiovascular condition of the living body based on the updated pulse wave signal.

In some embodiments, the processing module 530 may include a universal processor, e.g., a programmable logic device (PLD), an application-specific integrated circuit (ASIC), a microprocessor, a system on chip (SoC), a digital signal processor (DSP), or the like, or any combination thereof. Two or more of these universal processors in the processing module 530 may be integrated into a hardware device, or two or more hardware devices independently with each other. It should be understood, the universal processor in the processing module 530 may be implemented via various configurations. For example, the processing procedure of the processing module 530 may be implemented by hardware, software, or a combination of hardware software, not only by a hardware circuit in a programmable hardware device in an ultra large scale integrated circuit, a gate array chip, a semiconductor such a transistor, or a field programmable gate array, a programmable logic device, and also by a software performed by various processors, and also by a combination of the hardware and the software above (e.g., firmware).

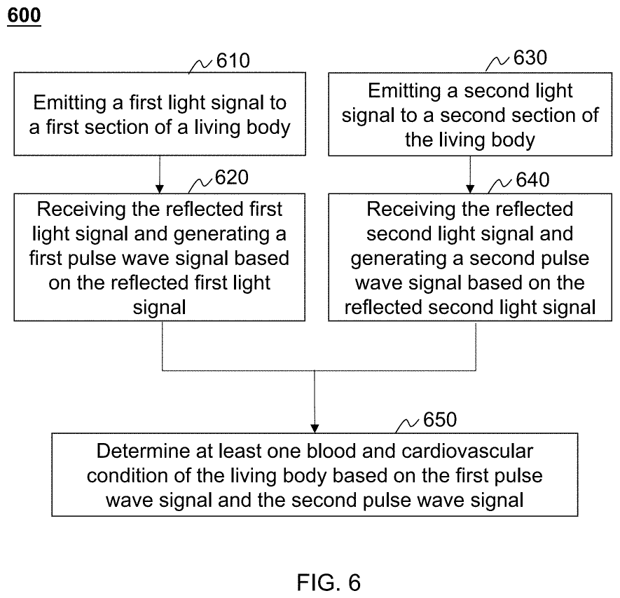

FIG. 6 is a flowchart illustrating an exemplary process for determining blood and cardiovascular condition according to some embodiments of the present disclosure. In some embodiments, the process 600 may be implemented as a set of instructions (e.g., an application) stored in the storage 390, ROM 240 or RAM 250. The blood and cardiovascular condition determination apparatus 110, the processor 230 and/or the MCU 340 may execute the set of instructions, and when executing the instructions, the blood and cardiovascular condition determination apparatus 110, the processor 230 and/or the MCU 340 may be configured to perform the process 600. The operations of the illustrated process present below are intended to be illustrative. In some embodiments, the process 600 may be accomplished with one or more additional operations not described and/or without one or more of the operations herein discussed. Additionally, the order in which the operations of the process as illustrated in FIG. 6 and described below is not intended to be limiting.

In 610, the blood and cardiovascular condition determination apparatus 110 (e.g., a first light emitter 410) may emit a first light signal to a first section of a living body. In some embodiments, the first light signal may have particular penetration characteristics, including a light wavelength, a diffusion angle, penetration depth, or the like, or any combination thereof.

In some embodiments, the light wavelength may be in a range of an infrared wavelength (e.g., 700 nm-1 mm), a red wavelength (e.g., 620-750 nm), a blue wavelength (e.g., 450-495 nm), an ultraviolet wavelength (e.g., 10 nm-400 nm), a green wavelength (e.g., 495-570 nm), or the like, or any combination thereof. In some embodiments, the diffusion angle may be determined based on type of the luminotron inside the light emitter. For example, a laser diode (LD) may have a small diffusion angle, while a light emitting diode (LED) may have a large diffusion angle. In some embodiments, the penetration depth may be determined based on the light wavelength. For example, the first light signal with a long wavelength (e.g., a red wavelength) may penetrate the skin and reach, for example, blood vessels deep beneath the skin, while the first light signal with a short wavelength (e.g., an ultraviolet wavelength) may penetrate the skin and reach capillaries shallower than the blood vessels beneath the skin. The first light signal may be reflected by the blood vessels or capillaries and received by a first light receiver (e.g., a light receiver 420).

In some embodiments, the first light signal emitted by the first light emitter may include a light signal at a single light wavelength. In some embodiments, the first light signal emitted by the first light emitter may include a plurality of light signals with same or different light wavelengths.

In some embodiments, the living body may refer to an individual that may be detected in the blood and cardiovascular condition determination system 100. For example, the living body may be a human, an animal, a part of the human (e.g., the head, the heart, a vitro tissue), or the like, or a combination thereof. The first section may include a skin surface, a blood vessel, a capillary of the living body, and/or a part of the living body (e.g., finger, wrist, arm, heart).

In 620, the blood and cardiovascular condition determination apparatus 110 (e.g., a first light receiver 420) may receive the reflected first light signal and generate a first pulse wave signal. More particularly, the first light receiver may perform a photoelectric conversion of the signal that converts an intensity of the reflected light signal to a voltage or current of the pulse wave signal.

In 630, the blood and cardiovascular condition determination apparatus 110 (e.g., a second light emitter 410) may emit a second light signal to a second section of a living body. The second light signal may have a particular penetration characteristic. In some embodiments, the penetration characteristic of the second light signal may have same as or different from the penetration characteristic of the first light signal. For example, the first light signal may have a long wavelength (e.g., in a range of infrared wavelength) and a high pulse velocity. The second light signal may have a low wavelength (e.g., in a range of a blue wavelength to an ultraviolet wavelength) and a pulse velocity lower than the first light signal. The second light emitter may work in a similar way as the first light emitter and the descriptions thereof are not repeated herein.

In 640, the blood and cardiovascular condition determination apparatus 110 (e.g., a second light receiver 420) may receive the reflected second light signal and generate a second pulse wave signal. More particularly, the second light receiver may perform a photoelectric conversion of the signal that converts an intensity of the reflected light signal to a voltage or current of the pulse wave signal. The second light receiver may work in a similar way as the first light receiver and the descriptions thereof are not repeated herein.

In 650, the blood and cardiovascular condition determination apparatus 110 (e.g., the MCU 480) or the server (the processing module 530) may determine at least one blood and cardiovascular condition of the living body based on the first pulse wave signal and the second pulse wave signal. As used herein, the blood and cardiovascular condition may include a blood condition, such as a blood sugar level, a blood oxygen level, or a blood viscosity, and a cardiovascular condition, such as a blood pressure, an arteriosclerosis, a blood vessel aging level, or an electrocardiogram.

In some embodiments, the at least one blood and cardiovascular conditions may be obtained by analyzing the first pulse wave signal and the second pulse wave signal, respectively. For example, the first blood and cardiovascular condition is the blood pressure and determined based on the first pulse wave signal. The second blood and cardiovascular condition is the blood oxygen and determined based on the second pulse wave signal.

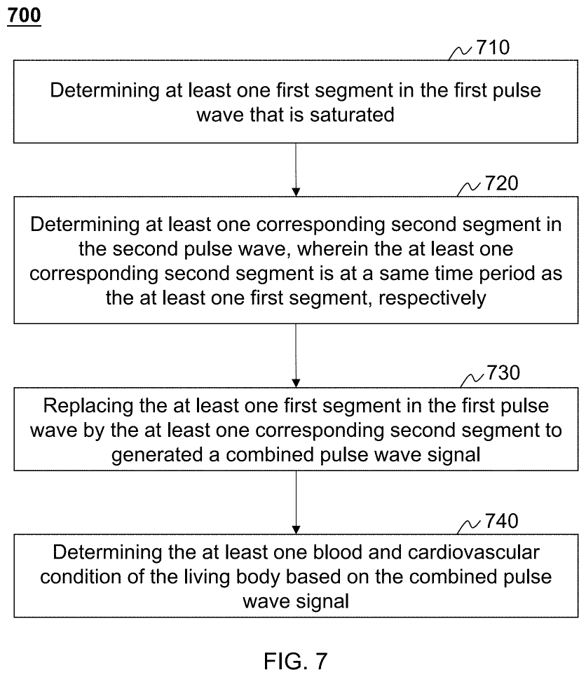

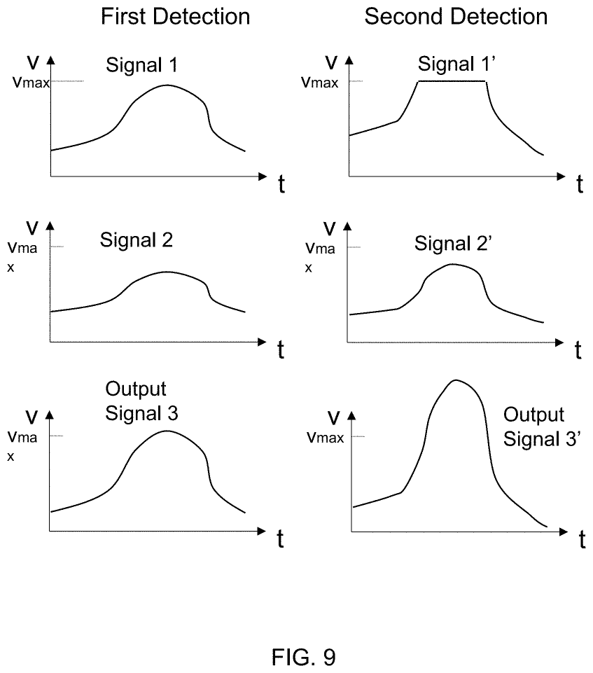

In some embodiments, the blood and cardiovascular condition may be obtained by analyzing the first pulse wave signal and the second pulse wave signal collectively. In some embodiments, a part of the first pulse wave signal may be saturated and replaced by a part of the second pulse wave signal to generate a combined pulse wave signal. The part of the first pulse wave signal and the part of the second pulse wave signal may be obtained at a same time period. Detailed descriptions of the generation of the combined pulse wave signal may be found elsewhere in this disclosure (e.g., FIGS. 7, 9 and the descriptions thereof).

In some embodiments, the blood and cardiovascular condition may be obtained based on an average pulse wave signal of the first pulse wave signal and the second pulse wave signal. In some embodiments, the first pulse wave signal and the second pulse wave signal may have a same weight. In some embodiments, the first pulse wave signal and the second pulse wave signal may have different weights (the average pulse wave signal becomes a weighted averaged pulse wave signal in this case). The different weights may be determined based on different signal-to-noise ratio (SNR) of the first pulse wave signal and the second pulse wave signal. In some embodiments, the weight may be proportional to the SNR. For example, the SNR ratio between the first pulse wave signal and the second pulse wave signal is 2:3, the weight of the first pulse wave signal is 0.4, and the weight of the second pulse wave signal is 0.6. The average pulse wave signal may be expressed as: 0.4*first pulse wave signal+0.6*second pulse wave signal.

In some embodiments, the first pulse wave signal and the second pulse wave signal may be corrected based on temperature information and motion information of the first section and the second section of the living body. Detailed descriptions of the correction may be found elsewhere in this disclosure (e.g., FIG. 8 and the descriptions thereof).

In some embodiments, the blood and cardiovascular condition determination apparatus 110 (e.g., a MCU 480) or the server (the processing module 530) may determine at least one blood and cardiovascular condition of the living body based on a biopotential signal of the living body (e.g., an ECG) and the pulse wave signals. The biopotential signal may be acquired by an ECG electrode.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations may be omitted in the exemplary process 600. For example, operations 630 and 640 may be omitted. The processing module 530 may determine at least one blood and cardiovascular condition of the living body merely based on the first pulse wave signal.

FIG. 7 is a flowchart illustrating an exemplary process for generating a combined pulse wave signal according to some embodiments of the present disclosure. In some embodiments, the process 700 may be implemented as a set of instructions (e.g., an application) stored in the storage 390, ROM 240 or RAM 250. The blood and cardiovascular condition determination apparatus 110, the processor 230 and/or the MCU 340 may execute the set of instructions, and when executing the instructions, the blood and cardiovascular condition determination apparatus 110, the processor 230 and/or the MCU 340 may be configured to perform the process 700. The operations of the illustrated process present below are intended to be illustrative. In some embodiments, the process 700 may be accomplished with one or more additional operations not described and/or without one or more of the operations herein discussed. Additionally, the order in which the operations of the process as illustrated in FIG. 7 and described below is not intended to be limiting. The process 700 may be performed after the first pulse wave signal and the second pulse wave signal are received in e.g., 620 and 640.

In 710, the blood and cardiovascular condition determination apparatus 110 (e.g., a MCU 480) or the server (the processing module 530) may determine at least one first segment in the first pulse wave that is saturated. As used herein, the segment being saturated may refer to that all values related to the segment are at a maximum voltage and cannot show the actual shape of the pulse wave detected in real-time. The saturation of the first segment of the first pulse wave may be due to design limitations of the light receiver or caused by, for example, a motion of the living body.

In 720, the blood and cardiovascular condition determination apparatus 110 (e.g., a MCU 480) or the server (the processing module 530) may determine at least one second segment in the second pulse wave signal corresponding to the at least one first segment, respectively. The at least one second segment may be at a same time period as the at least one first segment, respectively. In some embodiments, the shapes of the first pulse wave signal is similar to the second pulse wave signal but the first pulse wave signal may demonstrate higher measured values than the second pulse wave signal. As the first pulse wave signal may be saturated at a segment due to the high measurements, the second pulse wave signal is less likely to be saturated at a corresponding segment.

In 730, the blood and cardiovascular condition determination apparatus 110 (e.g., a MCU 480) or the server (the processing module 530) may replace the at least one first segment in the first pulse wave by the at least one corresponding second segment to generate a combined pulse wave signal. In some embodiments, the values related to the second segment in the second pulse wave signal that corresponds to the saturated segment in the first pulse wave signal may be multiplied by a factor so that the second segment is smoothly fit into the unsaturated segments in the first pulse wave signal. Detailed descriptions of the generation of the combined pulse wave signal may be found elsewhere in this disclosure (e.g., FIG. 9 and the descriptions thereof).

In 740, the blood and cardiovascular condition determination apparatus 110 (e.g., a MCU 480) or the server (the processing module 530) may determine the at least one blood and cardiovascular condition of the living body based on the combined pulse wave signal. The determination of the at least one blood and cardiovascular condition may be found similar to operation 650 and is not repeated herein.

FIG. 8 is a flowchart illustrating an exemplary process for updating a first pulse wave signal according to some embodiments of the present disclosure. In some embodiments, the process 800 may be implemented as a set of instructions (e.g., an application) stored in the storage 390, ROM 240 or RAM 250. The blood and cardiovascular condition determination apparatus 110, the processor 230 and/or the MCU 340 may execute the set of instructions, and when executing the instructions, the blood and cardiovascular condition determination apparatus 110, the processor 230 and/or the MCU 340 may be configured to perform the process 800. The operations of the illustrated process present below are intended to be illustrative. In some embodiments, the process 800 may be accomplished with one or more additional operations not described and/or without one or more of the operations herein discussed. Additionally, the order in which the operations of the process as illustrated in FIG. 8 and described below is not intended to be limiting. The process 800 may be performed after the first pulse wave signal is generated in 620 as a process for updating, correcting, or compensating the first pulse wave signal.

In 810, the blood and cardiovascular condition determination apparatus 110 (e.g., a MCU 480) or the server (the processing module 530) may receive a first pulse wave signal corresponding to a first section of a living body. Detailed descriptions of the generation of first pulse wave signal may be found elsewhere in this disclosure (e.g., 610, 630 in FIG. 6) and the descriptions thereof are not repeated herein.

In 820, the blood and cardiovascular condition determination apparatus 110 (e.g., a MCU 480) or the server (the processing module 530) may obtain a temperature of the first section of the living body. In some embodiments, the temperature may be measured by the temperature sensor 430.

In 830, the blood and cardiovascular condition determination apparatus 110 (e.g., a MCU 480) or the server (the processing module 530) may obtain a motion of the first section of the living body. In some embodiments, the motion may be detected by a motion sensor 450. The motion sensor 450 may be an accelerometer, gyroscope, gradiometer, or the like, any other motion sensing device. The motion of the section may include but not limited to moving from side to side, tilting upward and downward, tremoring, etc. and may be depicted by various parameters including a distance, a speed, a direction, an intensity, and/or a moving trace.

In 840, the blood and cardiovascular condition determination apparatus 110 (e.g., a MCU 480) or the server (the processing module 530) may determine whether the temperature is lower than a first temperature threshold (e.g., 5° C., 10° C., 30° C.) or higher than a second temperature threshold (e.g., 38° C., 45° C., 50° C.). In some embodiments, the first temperature threshold and the second temperature threshold may be the same (e.g., 36.5° C.). In response to a determination that the temperature is lower than the first temperature threshold or higher than the second temperature threshold, the process 800 may proceed to 850; otherwise, the process 800 may proceed to 860.

In 850, the temperature controller 440 may maintain the first section of the living body at a particular temperature (e.g., a normal temperature 36.5° C., the first temperature threshold, the second temperature threshold) or within a range of temperatures (e.g., a range between the first temperature threshold and the second temperature threshold). The temperature controller 440 may be a thermostat (e.g., a bimetal thermostat), a metal electrode, or the like, or any combination thereof. For example, when the blood and cardiovascular condition determination apparatus 110 determines that the temperature of the first section is lower than the first temperature threshold, the temperature controller 440 may heat the first section up till the normal temperature or the first temperature threshold.

In 860, the first pulse wave signal may be updated based on the temperature and/or the motion. In some embodiment, the updating of the first pulse wave signal may be determined based on a compensation algorithm. For example, the compensation algorithm includes a relationship between a compensation coefficient and a difference between the normal temperature (or default temperature when calculating the first pulse wave signal) of the living body and temperature of the first section or a relationship between a compensation coefficient and the motion. The first pulse wave signal may be updated based on the compensation coefficient. In some embodiments, the temperature obtained in 820 and the motion obtained in 830 may be used to directly update a blood and cardiovascular condition determined in e.g., 650 or 740.

FIG. 9 is a schematic diagram illustrating an exemplary method for generating a combined pulse wave signal. As shown in FIG. 9, signal 1, signal 2, and output signal 3 on the left may correspond to a first detection of pulse wave signals and signal 1′, signal 2′, and output signal 3′ on the right may correspond to a second detection of pulse wave signals. The signal 1 and the signal 1′ may be received from a first pulse wave signal sensor with respect to a first section of the living body. The signal 2 and signal 2′ may be received from a second pulse wave signal sensor with respect to a second section of the living body. The output signal 3 may be a combined pulse wave signal generated based on the signal 1 and the signal 2 and the output signal 3′ may be a combined pulse wave signal generated based on the signal 1′ and the signal 2′.

The first pulse wave signal sensor and the second pulse wave signal sensor may emit (and receive) light signals with similar penetration characteristics except that the light signal emitted by the first pulse wave signal sensor has higher intensity than the light signal emitted by the second pulse wave signal sensor. In the first detection, the signal 1 and the signal 2 show similar wave shapes due to their similar penetration characteristics. As the signal 1 is generated by a light signal with high intensity, the overall pulse wave of signal 1 demonstrates greater amplitude than that of the signal 2. In some embodiments, the signal-to-noise ratio (SNR) of signal 1 may be greater than the signal 2 and the signal 1 may be directly designated as the output signal in the first detection.

In the second detection, the signal 1′ is saturated, i.e., a segment (first segment) of the signal 1′ is at a maximum voltage and cannot show the real shape of the pulse wave. The saturation of signal 1′ may be caused by, for example, a motion of the living body. The signal 2′ is not saturated because signal 2′ is generated by a light signal with low intensity. Further, the signal 2′ shows the real shape of the pulse wave but is at a lower SNR than signal 1′. In order to obtain a pulse wave signal that shows the real shape of the pulse wave while maintaining the SNR as high as possible, signal 1′ and the signal 2′ may be combined to generate a combined pulse wave signal. More particularly, the unsaturated segment in the signal 1′ may be combined with the segment (second segment) in signal 2′ that corresponds to the saturated segment in the signal 1, i.e., the unsaturated segment of the signal 1′ is replaced by the corresponding segment of the signal 2′. In some embodiments, the second segment in signal 2′ that corresponds to the saturated segment in the signal 1′ may be multiplied by a factor so that the second segment in signal 2 is smoothly fit into the unsaturated segments in the signal 1′.

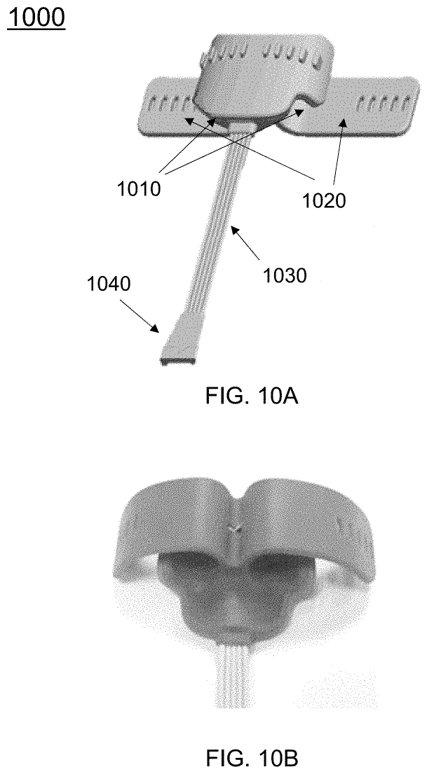

FIG. 10A is a schematic diagram illustrating a perspective view of an exemplary blood and cardiovascular condition determination apparatus according to some embodiments of the present disclosure. As shown in FIG. 10A, the blood and cardiovascular condition determination apparatus 1000 (i.e., the blood and cardiovascular condition determination apparatus 110 in FIG. 1) may include two straps 1020, and two pulse wave signal sensors 1010 configured on the inner sides of the straps 1020, respectively, connecting wires 1030 and a plug 1040.

In some embodiments, the pulse wave signal sensor 1010 may be a photo-plethysmography (PPG) signal sensor that receives light that has passed through or reflected by a section of a living body. Each of the two pulse wave signal sensor 1010 may include a light emitter (e.g., a LED) and a light receiver (e.g., a photoelectric receiver).

The strap 1020 may be wrapped around the section of the living body (e.g., a finger, an arm, a wrist). For example, two straps 1020 may respectively wrap two neighboring fingers or non-neighboring fingers of the living body to collect two separate pulse wave signals. The strap 1020 may be made of materials that enable the strap to be flexible to bend. In some embodiments, the length of each strap 1020 may be adjustable to fit different living bodies or different sections of the living body. For example, the strap 1020 may include a plurality of buttons to adjust the length such that the strap may match fingers in different sizes. In some embodiments, different parts in the strap 1020 may have different thickness. For example, the middle part of the strap 1020 may be thicker than two sides of the strap 1020 (the middle part of the strap 1020 may also be referred to as a main body). The middle part of the strap 1020 may contain one or more built-in devices (e.g., a temperature sensor, a motion sensor, a metal electrode, an ECG electrode, a processor, a battery, a wireless transceiver).

In some embodiments, the blood and cardiovascular condition determination apparatus 110 may further include a screen (not shown in the figure) on a surface of the blood and cardiovascular condition determination apparatus 1000. The screen may be a flat screen display or a bendable flexible display.

In some embodiments, the connecting wires 1030 and the plug 1040 may be connected to and exchange information with the processing module 530. In some embodiments, the plug 1040 may be connected with a power source to supply power to the blood and cardiovascular condition determination apparatus 1000. Data collected by the two pulse wave signal sensors 1010 may be transmitted to the terminal(s) 130 and the server 140 via the connecting wires 130. In some embodiments, the blood and cardiovascular condition determination apparatus 1000 may further include a built-in wireless communication module such as Bluetooth that enables the data exchange between the blood and cardiovascular condition determination apparatus 1000 and the terminal(s) 130 and/or server 140.

FIG. 10B is a diagram illustrating a bottom view of the exemplary blood and cardiovascular condition determination apparatus according to some embodiments of the present disclosure.

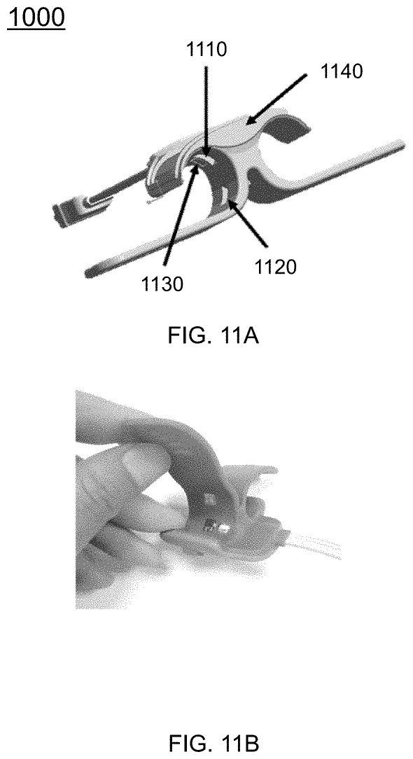

FIG. 11A is a schematic diagram illustrating lateral view of the exemplary blood and cardiovascular condition determination apparatus according to some embodiments of the present disclosure. The blood and cardiovascular condition determination apparatus 1000 may include an LED 1110, a photoelectric receiver 1120, a temperature sensor 1130 and a motion sensor 1140.

The LED 1110 may be configured on an inner surface of the blood and cardiovascular condition determination apparatus 1000. When the blood and cardiovascular condition determination apparatus 1000 starts to operate, the LED 1110 may emit a light signal passing through (and/or reflected by) a section of a living body. For example, the LED 1110 may emit a light signal with an infrared wavelength. The photoelectric receiver 1120 may be configured on the inner surface of the blood and cardiovascular condition apparatus 1000 at a side opposite to or the same as the LED 1110. The photoelectric receiver 1120 may receive the penetrated or reflected light signal 1110 and generate a pulse wave signal.

The temperature sensor 1130 may be configured on the inner surface of the blood and cardiovascular condition determination apparatus 1000. When the blood and cardiovascular condition determination apparatus 1000 is attached to a section of a living body, the temperature sensor 1130 is positioned to be, in contact with the section of the living body. The temperature sensor 1130 may measure the temperature of the living body to update the pulse wave signal based on a relationship between the temperature and the pulse wave signal.

The motion sensor 1140 may be placed in the middle part of the strap 1020 of the blood and cardiovascular condition determination apparatus 1000. The motion sensor 1140 may detect a motion of the living body. The motion sensor 1140 may be an accelerometer, gyroscope, gradiometer, or any other motion sensing device.

FIG. 11B is a diagram illustrating a side view of an exemplary blood and cardiovascular condition determination apparatus according to some embodiments of the present disclosure.

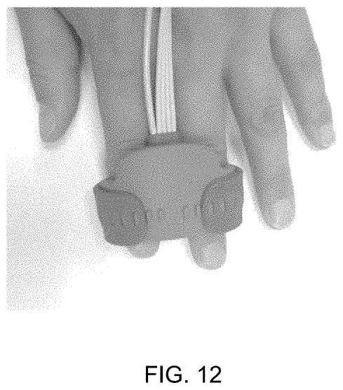

FIG. 12 is a diagram illustrating wearing view of an exemplary blood and cardiovascular condition determination apparatus according to some embodiments of the present disclosure. As shown in FIG. 12, when two straps (e.g., the straps 1020) of the blood and cardiovascular condition determination apparatus wrap two fingers respectively, the sensors configured on the inner sides of the blood and cardiovascular condition determination apparatus are in contact with the two fingers, for example, the pulse wave sensors, the temperature sensors, the temperature controller, and/or the ECG electrode may be in contact with the two fingers. The pulse wave signals may be collected from the two fingers and at least one blood and cardiovascular condition may be determined based on the pulse wave signals by the processor inside the blood and cardiovascular condition determination apparatus or the server 140.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment,” “one embodiment,” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “block,” “module,” “engine,” “unit,” “component,” or “system.” Furthermore, aspects of the present disclosure may take the form of a computer program product embodied in one or more computer-readable media having computer readable program code embodied thereon.

A computer readable signal medium may include a propagated data signal with computer readable program code embodied therein, for example, in baseband or as part of a carrier wave. Such a propagated signal may take any of a variety of forms, including electromagnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in a combination of one or more programming languages, including an object-oriented programming language such as Java, Scala, Smalltalk, Eiffel, JADE, Emerald, C++, C#, VB. NET, Python or the like, conventional procedural programming languages, such as the “C” programming language, Visual Basic, Fortran 1703, Perl, COBOL 1702, PHP, ABAP, dynamic programming languages such as Python, Ruby, and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer or in a cloud computing environment or offered as a service such as a software as a service (SaaS).

Furthermore, the recited order of processing elements or sequences, or the use of numbers, letters, or other designations, therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software-only solution—e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

1. A blood and cardiovascular condition determination apparatus, comprising:
a first pulse wave signal sensor configured to generate a first pulse wave signal including:
a first light emitter configured to emit a first light signal to a first section of a living body, wherein the first light signal is reflected by the first section; and
a first light receiver configured to receive the reflected first light signal and generate the first pulse wave signal based on the reflected first light signal;
a second pulse wave signal sensor configured to generate a second pulse wave signal including:
a second light emitter configured to emit a second light signal to a second section of the living body, wherein the second light signal is reflected by the second section; and
a second light receiver configured to receive the reflected second light signal and generate the second pulse wave signal based on the reflected second light signal; and
a processing unit, configured to determine at least one blood and cardiovascular condition of the living body based on the first pulse wave signal and the second pulse wave signal.

2. The blood and cardiovascular condition determination apparatus of claim 1, wherein the at least one blood and cardiovascular condition includes at least one of a blood pressure, a blood sugar level, a blood oxygen level, a blood vessel aging level, or a blood viscosity.

3. The blood and cardiovascular condition determination apparatus of claim 1, wherein:
the first light signal is configured to determine a first blood and cardiovascular condition of the living body; and
the second light signal is configured to determine a second blood and cardiovascular condition of the living body.

4. The blood and cardiovascular condition determination apparatus of claim 1, wherein the first light signal is configured to have higher intensity than the second light signal and the processing unit is further configured to:
determine at least one first segment in the first pulse wave that is saturated;
determine at least one corresponding second segment in the second pulse wave, wherein the at least one corresponding second segment is at a same time period as the at least one first segment, respectively;
replace the at least one first segment in the first pulse wave by the at least one corresponding second segment to generate a combined pulse wave signal; and
determine the at least one blood and cardiovascular condition of the living body based on the combined pulse wave signal.

5. The blood and cardiovascular condition determination apparatus of claim 1, wherein the processing unit is further configured to:
determine an average pulse wave signal based on the first pulse wave signal and the second pulse wave signal; and
determine the at least one blood and cardiovascular condition of the living body based on the average pulse wave signal.

6. The blood and cardiovascular condition determination apparatus of claim 5, wherein to determine the average pulse wave signal based on the first pulse wave signal and the second pulse wave signal, the processing unit is further configured to:
determine a first signal-to-noise ratio (SNR) of the first pulse wave signal and a second SNR of the second pulse wave signal;
determine a first weight of the first pulse wave signal and a second weight of the second pulse wave signal based on the first SNR and the second SNR; and
determine the average pulse wave signal based on the first pulse wave signal, the second pulse wave signal, the first weight, and the second weight.

7. The blood and cardiovascular condition determination apparatus of claim 1, wherein the first light signal includes at least two different wavelengths.

8. The blood and cardiovascular condition determination apparatus of claim 1, further comprising:
a temperature sensor configured to obtain a temperature of the first section when the first light signal is received,
wherein the processing unit is further configured to:
update the first pulse wave signal based on the temperature of the first tissue section to generate an updated first pulse wave signal; and
determine the at least one blood and cardiovascular condition of the living body based on the updated first pulse wave signal and the second pulse wave signal.

9. The blood and cardiovascular condition determination apparatus of claim 1, further comprising:
a temperature controller configured to maintain the temperature of the first section of the living body.

10. The blood and cardiovascular condition determination apparatus of claim 1, further comprising:
a motion sensor configured to obtain a motion of the first section when the first light signal is received,
wherein the processing unit is further configured to:
update the first pulse wave signal based on the motion of the first section to generate an updated first pulse wave signal; and
determine the at least one blood and cardiovascular condition of the living body based on the updated first pulse wave signal and the second pulse wave signal.

11. The blood and cardiovascular condition determination apparatus of claim 1, further comprising:
an electrocardiogram (ECG) electrode, configured to acquire a biopotential signal of the living body, and
wherein the processing unit is further configured to:
determine the at least one blood and cardiovascular condition of the living body based on the first pulse wave signal, the second pulse wave signal, and the biopotential signal.

12. The blood and cardiovascular condition determination apparatus of claim 1, wherein at least one of the first pulse wave signal sensor or the second pulse wave signal sensor is implemented on a wearable device attached to a finger of a human body.

13. The blood and cardiovascular condition determination apparatus of claim 1, further comprising:
a screen configured to display the at least one blood and cardiovascular condition.

14. The blood and cardiovascular condition determination apparatus of claim 1, further comprising:
a transceiver configured to transmit the at least one blood and cardiovascular condition to an electronic device.

15. A blood and cardiovascular condition determination method, implemented on a computing device having at least one storage device storing a set of instructions for determining at least one blood and cardiovascular condition and at least one processor in communication with the at least one storage device, the blood and cardiovascular condition determination method comprising:
generating, by a first pulse wave signal sensor, a first pulse wave signal, wherein generation of the first pulse wave signal includes:
emitting, by a first light emitter, a first light signal to a first section of a living body, wherein the first light signal is reflected by a first section; and
receiving, by a first light receiver, the reflected first light signal and generating the first pulse wave signal based on the reflected first light signal;
generating, by a second pulse wave signal sensor, a second pulse wave signal, wherein generation of the second pulse wave signal includes:
emitting, by a second light emitter, a second light signal to a second section of a living body, wherein the second light signal is reflected by a second section; and
receiving, by a second light receiver, the reflected second light signal and generating the second pulse wave signal based on the reflected second light signal; and
determining, by a processing unit, at least one blood and cardiovascular condition of the living body based on the first pulse wave signal and the second pulse wave signal.

16. (canceled)

17. (canceled)

18. The blood and cardiovascular condition determination method of claim 15, wherein the first light signal is configured to have higher intensity than the second light signal and the blood and cardiovascular condition determination method further includes:
determining at least one first segment in the first pulse wave that is saturated;
determining at least one corresponding second segment in the second pulse wave, wherein the at least one corresponding second segment is at a same time period as the at least one first segment, respectively;
replacing the at least one first segment in the first pulse wave by the at least one corresponding second segment to generated a combined pulse wave signal; and
determining the at least one blood and cardiovascular condition of the living body based on the combined pulse wave signal.

19. The blood and cardiovascular condition determination method of claim 15, further comprising:
determining an average pulse wave signal based on the first pulse wave signal and the second pulse wave signal; and
determining the at least one blood and cardiovascular condition of the living body based on the average pulse wave signal.

20. (canceled)

21. (canceled)

22. The blood and cardiovascular condition determination method of claim 15, further comprising:
obtaining, by a temperature sensor, a temperature of the first section when the first light signal is received;
updating the first pulse wave signal based on the temperature of the first tissue section to generate an updated first pulse wave signal; and
determining the at least one blood and cardiovascular condition of the living body based on the updated first pulse wave signal and the second pulse wave signal.

23. (canceled)

24. The blood and cardiovascular condition determination method of claim 15, further comprising:
obtaining, by a motion sensor, a motion of the first section when the first light signal is received;
updating the first pulse wave signal based on the motion of the first section to generate an updated first pulse wave signal; and
determining the at least one blood and cardiovascular condition of the living body based on the updated first pulse wave signal and the second pulse wave signal.

25-28. (canceled)

29. A non-transitory computer-readable storage medium storing instructions that, when executed by at least one processor of a system, cause the system to perform a blood and cardiovascular condition determination method, the blood and cardiovascular condition determination method comprising:
generating, by a first pulse wave signal sensor, a first pulse wave signal, wherein generation of the first pulse wave signal includes:
emitting, by a first light emitter, a first light signal to a first section of a living body, wherein the first light signal is reflected by a first section; and
receiving, by a first light receiver, the reflected first light signal and generating the first pulse wave signal based on the reflected first light signal;
generating, by a second pulse wave signal sensor, a second pulse wave signal, wherein generation of the second pulse wave signal includes:
emitting, by a second light emitter, a second light signal to a second section of a living body, wherein the second light signal is reflected by a second section; and
receiving, by a second light receiver, the reflected second light signal and generating the second pulse wave signal based on the reflected second light signal; and
determining, by a processing unit, at least one blood and cardiovascular condition of the living body based on the first pulse wave signal and the second pulse wave signal.
